(19) United States
(12) Patent Application Publication
(10) Pub. No.: US 2005/0118674 A1
(43) Pub. Date: Jun. 2, 2005

(54) Engineering of Controlled Deamidation Rates in Peptides, Proteins, and Similar Structures

(76) Inventor: Noah E. Robinson, Cave Junction, OR (US)

Correspondence Address: Noah E. Robinson, 2251 Dick George Rd., Cave Junction, OR, 97523, US

(21) Appl. No.: 10/707,262

(22) Filed: Dec. 2, 2003

(51) Int. Cl. C12Q 1/68; C07H 21/04; C07K 14/47

(52) U.S. Cl. 435/69.1; 435/320.1; 435/325; 530/350; 530/399; 536/23.5

(57) ABSTRACT

Experiments that quantitatively determine the sequence dependence of deamidation and three-dimensional structure observations have been carried out. These experiments and theoretical computation methods based upon them, allow the invention of techniques for engineering of deamidation rates for amides in peptides, hormones and proteins as well as peptide-like, hormone-like and protein-like molecules. Modification of the amide, the residues or residue-like structures on either side of the amide or of other structural parameters can be carried out. This allows the stabilization of amides, the destabilization of amides, or the setting of amides to specific rates for use in engineering of molecules for pharmaceutical, industrial or other purposes. This work is also applied to the isomerization and racemization of carboxylic acids in similar ways.

### CROSS-REFERENCE TO RELATED APPLICATIONS

Application Ser. No. 10/707,263. Design Technique for Use in Engineering of Deamidation Rates of Peptides, Proteins, Hormones, and Peptide-Like, Protein-Like and Hormone-Like Molecules.

### STATEMENT REGARDING FEDERALLY SPONSORED RESEARCH OR DEVELOPMENT

Not Applicable

### INCORPORATION-BY-REFERENCE OF MATERIAL SUMBMITTED ON A COMPACT DISC

Enclosed CD of book: Molecular Clocks: Deamidation of Asparaginyl and Glutaminyl Residues in Peptides and Proteins, Robinson, N. E. and Robinson, A. B., Althouse Press, Cave Junction, Oreg., ISBN 1-59087-250-9. This 448 page book contains a complete review of the subject, including over 1785 references to the research literature, 86 Figures and 16 Tables. The inventions described in this patent are placed in context by this book.

### BACKGROUND OF THE INVENTION

The deamidation of peptides and proteins as well as molecules related to peptides and proteins is a well known phenomenon. In this reaction, Asn or Gln residues are gradually changed into Asp and Glu residues and their isomers respectively. The rate of this reaction is dependent on the primary sequence, three-dimensional structure, pH, temperature, buffer type, ionic strength and other solution properties. The half-time varies from less than 1 day to more than a century. The reaction introduces a negative charge into the molecule. In addition, the isomerization products β-Asp and β-Glu as well D-isomerized forms and chain cleavage also accompany the reaction.

The stability of Asn and Gln in pharmaceutical and other types of commercial preparations is a major field of study. Efforts have been made to discover formulation conditions that will minimize the rate of deamidation of amides in these preparations. There is also commercial potential in induced or controlled deamidation as an active aspect of the product.

### BRIEF SUMMARY OF THE INVENTION

For the purposes of this work the definition of terms is as follows: Asn—Asparaginyl residue in a peptide or protein, Gln—Glutaminyl residue in a peptide or protein.

The inventions described here pertain to the engineering of peptides, hormones, and proteins as well as peptide-like, hormone-like and protein-like molecules.

It is well known that for peptide sequences of the type AsnXxx and GlnXxx, where Xxx is any natural or unnatural amino acid, the rate of deamidation of either Asn or Gln depends very strongly on the identity of Xxx. These results are applicable to peptides, proteins and hormones as well as any amide-containing molecule with similar structure. It is also applicable to isomerization of AspXxx and GluXxx sequences.

I have done extensive work showing the quantitative sequence dependence of these reactions. I have also invented a method for applying this sequence dependence to proteins, peptides, and other similar molecules, in conjunction with their three-dimensional structures.

These inventions allow the prediction of deamidation rates of amides as a function of primary and three-dimensional structure, if the three-dimensional structures is known. They also provide quantitative information about the parameters that make up these rates and show which structural elements are important for each rate.

These inventions can be used to modify predictably structural elements to provide stability or controlled instability in amides or acids in pharmaceutical and other types of commercial preparations. Specifically there are three major types of modifications that can be made that will change the rate by amounts that can be quantitatively or qualitatively determined from these inventions. Asp and Glu residues also undergo reactions controlled in this way.

1. Modification of the residue or residue-like structure to the carboxyl-side or amino-side of the amides or acids. This can be done by substitution of a different natural or non-natural amino acid side chain.

2. Exchange of Asn for Gln or Gln for Asn. Gln deamidation and probably Glu isomerization is substantially slower by a quantitative amount.

3. Modification of other surrounding structural elements that affect the rate of the reaction as determined by my current three-dimensional calculation procedure or a similar procedure resulting from improvements in the current method.

These inventions allow the engineering of molecules with specific amide structures that will deamidate at specified rates. These procedures can be used to design stable and unstable forms for pharmaceutical, industrial, and other products. This can be used to increase the shelf-life of such products through minor modifications, prevent or at least slow down the gradual formation of impurities in preparations with these modifications, and may make possible as a result of minor modifications the use of products that would otherwise be too unstable for practical purposes. The engineering of products with unstable amides that are programmed to deamidate at specific rates is also a valuable application of this procedure.

### BRIEF DESCRIPTION OF THE SEVERAL VIEWS OF THE DRAWING(S)

Not Applicable

### DETAILED DESCRIPTION OF THE INVENTION

General Method:

It was known before the invention of the method described here, that the sequence and structure around an amide has a large effect on the deamidation rate. Unknown, however, was the necessary quantitative information that would allow engineering of stable amides or amides with predetermined rates based on modification of the sequence and three-dimensional structure.

Experiments which I carried out showed quantitatively the effects of sequence dependent deamidation. One of the discoveries made was that the sequence dependence of deamidation is much richer and covers a wider range than previously thought. In 37° C., pH 7.4, 0.15 M Tris buffer, the combination of XxxAsnYyy and XxxGlnYyy sequences where Xxx and Yyy are any of the naturally occuring amino acids covers a range from less than 1 day to over 15,000 days with the entire range in between available.

In addition to the sequence dependent work, I have also invented methods that allow application of this sequence dependent data to three-dimensional protein structures to permit the prediction of protein deamidation rates. This method is applicable to any peptide type structures including peptides, hormones, and proteins and peptide-like, hormone-like, and protein-like molecules, as well as similar structures that deamidate in the same way.

This prediction procedure is based on identifying structural elements in a protein or similar molecule that contribute to the rate in known quantitative ways. These include, but are not limited to, hydrogen bonds of various types, disulfide bonds, alpha-helices, and beta-sheets. The effect of each structure depends on a variety of quantitative factors.

The invention of these prediction techniques had never been attempted before. Not only do they allow prediction of deamidation rates to very high reliability, but the calculation shows what structural features are responsible for each particular rate and what changes would be necessary to modify the rate in a quantitative manner.

Sequence Dependence:

Tables 1 and 2 show the sequence dependence of deamidation measured using natural amino acid variations in pentapeptides. Non-natural variations provide an even greater range of sequences to choose from.

Table 1 describes the sequence dependence of Asn sequences. It is based on pentapeptide rates measure in 37° C., pH 7.4, 0.15 M Tris buffer. The applicability of a pentapeptide model to sequence dependence was verified in a separate set of experiments. All values listed in this table are experimental except for the four values in boxes, which were estimated from the rest of the data.

TABLE 1
First-Order Deamidation Halftimes of GlyXxxAsnYyyGly in days at pH 7.4, 37.0° C., 0.15 M Tris HCl

| Xxx\Yyy | Gly | His | Ser | Ala | Asp | AmCys | Thr | Cys | Lys | Met | Glu | Arg |
|---|---|---|---|---|---|---|---|---|---|---|---|---|
| Gly | 1.03 | 9.2 | 11.8 | 21.1 | 28.0 | 27.6 | 39.8 | 40.6 | 48.2 | 50.4 | 73.9 | 57.8 |
| Ser | 0.96 | 9.0 | 15.1 | 24.1 | 30.3 | 41.3 | 45.7 | 60.2 | 55.5 | 54.9 | 59.7 | 59.7 |
| Thr | 1.04 | 9.6 | 17.1 | 24.6 | 27.9 | 34.4 | 50.0 | 55.5 | 57.6 | 47.6 | 60.8 | 51.2 |
| Cys | 1.14 | 10.8 | 19.0 | 26.4 | 30.6 | 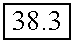 | 48.7 | 46.0 | 46.6 | 64.5 | 48.3 | 83.1 |
| AmCys | 1.14 | 10.9 | 15.4 | 21.5 | 32.9 | 39.3 | 41.7 | 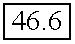 | 48.9 | 56.5 | 45 | 58.8 |
| Met | 1.04 | 10.2 | 15.2 | 22.1 | 26.4 | 33.8 | 43.6 | 49.6 | 60.4 | 56.9 | 72.4 | 58.8 |
| Phe | 1.15 | 10.2 | 18.1 | 24.2 | 27.4 | 29.8 | 39.0 | 46.5 | 58.2 | 58.6 | 62.4 | 61.2 |
| Tyr | 1.49 | 10.2 | 11.9 | 24.3 | 28.4 | 33.3 | 38.1 | 48.6 | 55.1 | 64.3 | 41.0 | 56.9 |
| Asp | 1.53 | 9.7 | 17.0 | 24.0 | 29.4 | 45.8 | 52.4 | 54.1 | 75.9 | 57.3 | 46.8 | 87.2 |
| Glu | 1.45 | 9.0 | 16.4 | 25.8 | 32.0 | 32.1 | 36.8 | 44.2 | 77.8 | 59.6 | 60.3 | 80.9 |
| His | 1.14 | 10.7 | 15.7 | 24.6 | 31.2 | 33.8 | 47.2 | 43.9 | 50.2 | 63.1 | 69.4 | 48.9 |
| Lys | 1.02 | 10.5 | 15.6 | 23.6 | 34.0 | 36.5 | 58.1 | 49.0 | 53.5 | 60.9 | 72.5 | 57.4 |
| Arg | 1.00 | 10.0 | 14.3 | 24.4 | 34.7 | 42.3 | 50.7 | 50.5 | 49.6 | 74.4 | 68.3 | 67.4 |
| Ala | 1.05 | 9.3 | 14.9 | 22.5 | 31.9 | 40.6 | 43.5 | 63.7 | 55.9 | 59.2 | 74.1 | 62.4 |
| Leu | 1.08 | 10.7 | 16.7 | 25.1 | 32.1 | 33.6 | 46.1 | 53.5 | 60.1 | 62.6 | 56.7 | 62.1 |
| Val | 1.23 | 10.2 | 18.2 | 27.5 | 33.5 | 34.7 | 49.9 | 63.2 | 63.8 | 65.7 | 64.8 | 67.4 |
| Ile | 1.26 | 11.5 | 14.5 | 25.9 | 33.8 | 33.0 | 46.3 | 52.7 | 64.4 | 58.8 | 58.6 | 66.4 |
| Trp | 1.75 | 11.3 | 15.5 | 30.7 | 43.6 | 42.9 | 38.9 | 83.1 | 59.4 | 64.2 | 75.7 | 73.9 |
| Pro | 1.18 | 12.8 | 18.9 | 31.8 | 48.6 | 43.7 | 63.1 | 60.0 | 67.8 | 78.4 | 92.0 | 72.9 |
| Mean | 1.19 | 10.3 | 15.9 | 25.0 | 32.5 | 36.7 | 46.3 | 53.2 | 58.4 | 60.9 | 63.3 | 65.0 |
| St. Dev. | 0.05 | 0.23 | 0.49 | 0.67 | 1.3 | 1.2 | 1.7 | 2.4 | 2.1 | 1.8 | 3.1 | 2.5 |
| % St. Dev. | 4.4 | 2.2 | 3.1 | 2.7 | 4.1 | 3.3 | 3.6 | 4.5 | 3.6 | 2.9 | 4.8 | 3.9 |
| Median | 1.14 | 10.2 | 15.6 | 24.4 | 31.9 | 34.7 | 46.1 | 50.5 | 57.6 | 59.6 | 62.4 | 62.1 |

| Xxx\Yyy | Phe | Tyr | Trp | Leu | Val | Ile | Pro | Median† |
|---|---|---|---|---|---|---|---|---|
| Gly | 64.0 | 63.6 | 77.1 | 104 | 224 | 287 | 7170 | 50.4 |
| Ser | 52.2 | 64.7 | 76.8 | 110 | 233 | 285 | 7060 | 55.5 |
| Thr | 76.4 | 80.6 | 72.5 | 110 | 237 | 279 | 6290 | 55.5 |
| Cys | 73.9 | 83.9 | 111 | 119 | 229 | 304 | 1550 | 48.7 |
| AmCys | 63.3 | 78.8 | 81.3 | 100 | 215 | 250 | 3900 | 48.9 |
| Met | 61.9 | 74.0 | 92.7 | 113 | 211 | 275 | 9300 | 57.9 |
| Phe | 69.5 | 75.1 | 102 | 118 | 203 | 287 | 7990 | 58.6 |
| Tyr | 58.0 | 70.6 | 120 | 118 | 241 | 306 | 9830 | 51.8 |
| Asp | 70.1 | 70.4 | 80.3 | 111 | 241 | 298 | 11800 | 55.7 |
| Glu | 70.2 | 94.5 | 98.4 | 130 | 268 | 279 | 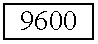 | 59.9 |
| His | 72.1 | 82.3 | 95.4 | 116 | 247 | 327 | 8440 | 50.2 |
| Lys | 70.1 | 96.7 | 98.1 | 119 | 246 | 313 | 4940 | 58.1 |
| Arg | 68.3 | 90.0 | 127 | 128 | 247 | 311 | 5790 | 67.4 |
| Ala | 65.6 | 73.9 | 130 | 124 | 254 | 300 | 7370 | 62.4 |
| Leu | 72.4 | 75.7 | 74.5 | 155 | 294 | 391 | 10500 | 60.1 |
| Val | 66.6 | 79.2 | 88.9 | 154 | 291 | 366 | 8030 | 64.8 |
| Ile | 61.5 | 79.3 | 86.7 | 154 | 295 | 384 | 11600 | 58.8 |
| Trp | 71.1 | 92.6 | 135 | 133 | 226 | 286 | 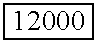 | 67.6 |
| Pro | 100 | 114 | 122 | 181 | 364 | 455 | 6590 | 72.9 |
| Mean | 68.8 | 81.1 | 98 | 126 | 251 | 315 | 7000 | 60.9 |
| St. Dev. | 2.3 | 3.0 | 4.9 | 5.1 | 9.3 | 12.2 | 600 | 2.3 |
| % St. Dev. | 3.4 | 3.7 | 5.0 | 4.0 | 3.7 | 3.9 | 8.8 | 3.7 |
| Median | 69.5 | 79.2 | 95 | 119 | 241 | 300 | 7100 | 59.6 |

†Median does not include Yyy = AmCys
Bold type values are experimental

Table 2 describes the sequence dependence of Gln peptides. It is also based on pentapeptide rates measure in 37° C., pH 7.4, 0.15 M Tris buffer. In this case, the 52 values shown in bold were measured, and the rest of the values were derived from surface fitting.

Tables 1 and 2 were published in: Robinson, N. E., Robinson, Z. W., Robinson, B. R., Robinson, A. L., Robinson, J. A., Robinson, M. L., and Robinson, A. B., (May 2004) Structure-dependent nonenzymatic deamidation of glutaminyl and asparaginyl pentapeptides, J. Peptide Res., 63, 426-436.

TABLE 2
First-Order Deamidation Haiftimes of GlyXxxGlnYyyGly in days at pH 7.4, 37.0 ° C., 0.15 M Tris HCl

| Xxx\Yyy | Gly | Cys | Met | Thr | Ser | Ala | His | Lys | Leu | Ile |
|---|---|---|---|---|---|---|---|---|---|---|
| Cys | 560 | 800 | 3200 | 3500 | 3800 | 4100 | 4200 | 4400 | 4800 | 4900 |
| Met | 600 | 900 | 3500 | 3800 | 4100 | 4400 | 4400 | 4600 | 5000 | 5000 |
| Thr | 670 | 1000 | 3700 | 4000 | 4200 | 4300 | 4500 | 4800 | 5200 | 5300 |
| Lys | 650 | 1000 | 4000 | 4100 | 4200 | 4300 | 6100 | 4000 | 5300 | 5400 |
| Arg | 660 | 1000 | 4100 | 4200 | 4300 | 4400 | 4900 | 4000 | 5400 | 5500 |
| Val | 640 | 1300 | 4200 | 4300 | 4400 | 4500 | 5000 | 5200 | 5500 | 5600 |
| Pro | 630 | 1600 | 4500 | 4600 | 4600 | 4700 | 5200 | 5500 | 5800 | 6000 |
| Ala | 610 | 1900 | 4400 | 5100 | 5200 | 5300 | 5500 | 5700 | 6100 | 6200 |
| Gly | 650 | 1900 | 4500 | 5200 | 5700 | 5900 | 5900 | 6000 | 6200 | 6300 |
| Leu | 670 | 2000 | 4800 | 5300 | 5800 | 6000 | 6100 | 6100 | 6300 | 6500 |
| Ile | 620 | 2000 | 5100 | 5300 | 5800 | 6200 | 6100 | 6100 | 6300 | 6500 |
| Phe | 660 | 2000 | 5100 | 5300 | 5900 | 6300 | 6200 | 6200 | 6400 | 6400 |
| Ser | 700 | 2100 | 5100 | 5400 | 6000 | 6400 | 6500 | 6300 | 6100 | 5900 |
| Glu | 750 | 2100 | 5200 | 5400 | 6100 | 7100 | 2500 | 4600 | 4300 | 4200 |
| Asp | 800 | 2100 | 5200 | 5400 | 6200 | 7100 | 2500 | 4600 | 6200 | 6400 |
| His | 850 | 2200 | 5200 | 5500 | 6300 | 7200 | 7200 | 4000 | 6600 | 6700 |
| Tyr | 850 | 2200 | 5300 | 5600 | 6400 | 7300 | 7400 | 7500 | 7800 | 7900 |
| Trp | 850 | 2300 | 5300 | 5600 | 6500 | 7400 | 7500 | 7600 | 7900 | 8000 |
| Mean | 690 | 1700 | 4600 | 4900 | 5300 | 5700 | 5400 | 5400 | 6000 | 6000 |
| St. Dev. | 22 | 129 | 163 | 169 | 228 | 296 | 352 | 272 | 226 | 233 |
| % St. | 3.2 | 7.6 | 3.5 | 3.4 | 4.3 | 5.2 | 6.5 | 5.0 | 3.8 | 3.9 |
| Dev. | | | | | | | | | | |
| Median† | 660 | 1950 | 4650 | 5250 | 5750 | 5950 | 6000 | 6050 | 6250 | 6400 |

| Xxx\Yyy | Val | Arg | Glu | Asp | Phe | Pro | Tyr | Trp | Median |
|---|---|---|---|---|---|---|---|---|---|
| Cys | 5000 | 5100 | 5600 | 6100 | 6500 | 7100 | 7900 | 9100 | 4800 |
| Met | 5000 | 5100 | 5800 | 6200 | 6600 | 7300 | 8200 | 9400 | 5000 |
| Thr | 5100 | 5100 | 5900 | 6300 | 6800 | 7500 | 8400 | 9700 | 5100 |
| Lys | 5700 | 2300 | 5400 | 5900 | 7000 | 7700 | 8800 | 10000 | 5300 |
| Arg | 5800 | 2300 | 5400 | 5900 | 7100 | 8100 | 9200 | 11000 | 4900 |
| Val | 5900 | 6100 | 6500 | 7000 | 7200 | 8500 | 9700 | 12000 | 5500 |
| Pro | 6200 | 6400 | 6800 | 7200 | 7300 | 8900 | 10000 | 13000 | 5800 |
| Ala | 6400 | 7200 | 7300 | 7400 | 7500 | 9300 | 10000 | 14000 | 6100 |
| Gly | 6500 | 7200 | 7300 | 7600 | 7600 | 10000 | 12000 | 15000 | 6200 |
| Leu | 6800 | 7200 | 7400 | 7800 | 8000 | 10000 | 12000 | 16000 | 6300 |
| Ile | 7100 | 7200 | 7700 | 8100 | 8100 | 10000 | 12000 | 16000 | 6300 |
| Phe | 7100 | 7200 | 8100 | 8200 | 8200 | 10000 | 12000 | 16000 | 6400 |
| Ser | 6800 | 7200 | 8100 | 8200 | 8300 | 10000 | 13000 | 17000 | 6400 |
| Glu | 6400 | 5200 | 8200 | 8300 | 8400 | 10000 | 13000 | 17000 | 5400 |
| Asp | 6600 | 5200 | 8200 | 8400 | 8500 | 11000 | 13000 | 17000 | 6200 |
| His | 6800 | 4500 | 5800 | 5600 | 8600 | 11000 | 14000 | 18000 | 6300 |
| Tyr | 8000 | 8100 | 8300 | 8600 | 8700 | 11000 | 14000 | 18000 | 7800 |
| Trp | 8200 | 8300 | 8500 | 8800 | 8600 | 11000 | 14000 | 19000 | 7900 |
| Mean | 6400 | 5900 | 7000 | 7300 | 7700 | 9400 | 11200 | 14300 | 6000 |
| St. Dev. | 221 | 423 | 273 | 259 | 180 | 329 | 521 | 809 | 246 |
| %St. Dev. | 3.4 | 7.2 | 3.9 | 3.5 | 2.3 | 3.5 | 4.7 | 5.7 | 4 |
| Median† | 6650 | 7200 | 7350 | 7700 | 7800 | 10000 | 12000 | 15500 | 6150 |

†Median without charged residues.
Bold type values are experimental.

Deamidation rates are affected by a wide variety of parameters, including, pH, Temperature, Ionic Strength, and Buffer Ions. These rates are measured under pH and Temperature conditions that are applicable to biological systems. The buffer type and concentration were chosen to minimize ion affects to the extent possible given the experimental limitations. Modification of these conditions will change the rates in Tables 1 and 2. As long as the conditions are not taken to extremes (i.e. high temperature, or strongly acidic or basic conditions) the qualitative sequence dependence should remain the same and the rates reported here can be used with necessary adjustments.

It is also clear that direct hydrolysis of Gln and Asn take place in addition to the regular sequence dependent mechanism. This hydrolysis is sequence dependent as well, but an average value of about an 8000 day half-time can be taken as a rough approximation based on this and other data measured at the same time. This does not effect the Asn rates significantly, but is responsible for the leveling off of the Gln rates at around this level. This hydrolysis is also effected by the reaction conditions.

The sequence dependence apparent in Tables 1 and 2 is of great value in engineering stable amides, unstable amides, or amides with particularly desired rates. Isomerization of acid residues will follow a very similar sequence dependence, offset by a determinable amount.

Gln vs. Asn Deamidation:

It is apparent from the data shown in Tables 1 and 2 that the deamidation rates of Asn and Gln cover markedly different ranges. One of the discoveries in these experiments was that their sequence dependencies are complementary. Asn sequences cover the range from about 1 day to 450 days. Gln picks up at 560 days and carries these rates out to tens of thousands of days.

This opens up a new possibility for engineering of amide rates. It is possible to switch half-time ranges simply by substituting Asn for Gln or Gln for Asn depending on the desired effect. In many cases where it is desirable to introduce or leave in place an amide, the difference of one CH₂ group in chain length may not be critical.

Moreover, the fact that this range switching can be done raises another possibility. Other modifications of Gln and Asn may lie in different ranges. Thus the substitution of unnatural amide side-chains is also a valuable procedure.

Three-Dimensional Effects of Deamidation:

The invention of the three-dimensional prediction method for deamidation rates has been developed in two phases. The first of these was the invention of a technique for determining deamidation rates in proteins based on manually counting the number of each type of structure that can affect the rate. Each of these effects is then summed with special coefficients to produce the correct rate. The procedure was calibrated on known relative deamidation rates and then found to be quite accurate in predicting absolute rates.

Secondly, the procedure was adapted to an automated method by means of an extensive C++ program. Some modifications were made when this was done, but the basic procedure remained the same.

I am not attempting to patent this C++ program. There are many ways to write such programs and the current version is protected by copyright. What is being patented is the method used to write it which is based on the manual procedure and minor modifications and improvements that are particularly adapted to computerized calculation and include many conceptual innovations.

It will be obvious to anyone who studies and understands these methods that there are variations in the procedure and even some improvements that could be made which would yield similar results. Any such modifications are understood to be products of this invention and come under the scope of this patent.

The deamidation coefficient, C_{D}, for and amide is defined as: C_{D}=(0.01)(t−_{p1/2})(e^{f(Cm, CSn, Sn)}).

Here t_{1/2} is the pentapeptide primary structure half life, Cₘ is a structure proportionality factor, C_{Sn} is the 3D structure coefficient for the nth structure observation, Sₙ is that observation, and f(Cₘ, C_{Sn}, Sₙ)=Cₘ[(C_{S1})(S₁)+(CS₂)(S₂)+(CS₃)(S₃)−(C_{S4,5})(S₄)/(S₅)+(C_{S6})(S₆)+(C_{S7})(S₇)+(C_{S8})(S₈)+(C_{S9})(S₉)+(C_{S10})(1−S₁₀)+(C_{S11})(5-S₁₁)+(C_{S12})(5−S₁₂)]. The structure observations, Sₙ, were selected as those most likely to impede deamidations, including hydrogen bonds, α-helices, β-sheets, and peptide inflexibilities. The functional form of C_{D} assumes that each of these structural factors is added to the reaction activation energy. The observed Sₙ were:

For Asn in an α-helical region:

S₁=distance in residues inside the α-helix from the NH₂ end, where S₁=1 designates the end residue in the helix, 2 is the second residue, and 3 is the third. If the position is 4 or greater, S₁=0.
S₂=distance in residues inside the α-helix from the COOH end, where S₁=1 designates the end residue in the helix, 2 is the second residue, and 3 is the third. If the position is 4 or greater or S₁≠0, then S₂=0.
S₃=1 if Asn is designated as completely inside the α-helix, because it is 4 or more residues from both ends. If the Asn is completely inside, S₃=1, S₁=0, and S−₂=0. If S₁≠0 or S₂≠0, then S₃=0.

For flexibility of a loop including Asn between two adjacent antiparallel βsheets:
S₄=number of residues in the loop.
S₅=number of hydrogen bonds in the loop. S_{S}≧1 by definition.

For hydrogen bonds:
S₆=the number of hydrogen bonds to the Asn side chain C═O group. Acceptable values are 0, 1, and 2.
S₇=the number of hydrogen bonds to the Asn side chain NH₂ group. Acceptable values are 0, 1, and 2.
S₈=the number of hydrogen bonds to the backbone nitrogen atom in the peptide bond on the COOH side of Asn. Hydrogen bonds counted in S₆ or S₇ are not included. Acceptable values are 0 and 1. This nitrogen atom is used in the five-membered succinimide ring.

S₉=additional hydrogen bonds, not included in S₆, S₇, and S₈, that would need to be broken to form the succinimide ring.

For Asn situated so that no α-helix, β-sheet, or disulfide bridge structure is between the Asn and the end of the peptide chain:

S₁₀=1 if the number of residues between the Asn and the nearest such structure is 3 or more. If the number of intervening residues is 2, 1, or 0, or Asn not between structure and chain end, then S₁₀=0.

If the Asn lies near to any α-helix, β-sheet, or disulfide bridge structures:
S₁₁=the number of residues between the Asn and the structure on the NH₂ side, up to a maximum of 5. Values of 0, 1, 2, 3, 4, and 5 are acceptable.
S₁₂=the number of residues between the Asn and the structure on the COOH side, up to a maximum of 5. Values of 0, 1, 2, 3, 4, and 5 are acceptable.

Hydrogen bonds selected by the Swiss Protein Data Bank (PDB) viewer were accepted if the bond length was 3.3 Å or less and there was room in the structure to accommodate the van der Waals radius of the hydrogen. In the computerized procedure this bond length was optimized at 4.1 Å, and the bond angles and number of bonds per atom were adjusted to physically correct and optimized values. The Swiss PDB viewer, according to the customary criteria, selected α-helices and β-sheets. All primary structure t_{1/2} values were those published⁶, except for Asn with carboxyl-side Pro, Asn, or Gln and N-glycosylated Asn. Estimated values were used for any sequence for which the primary sequence rate was not known.

Coefficients Used in Equation:
C_{D} values (“Coefficient of Deamidation”) were optimized by using various values for Cₘ and C_{Sn} to maximize the value of the deamidation resolving power, D_{P}, as described in the calibration procedure section. The optimized values were Cₘ=0.48, C−_{S1}=1.0, C_{S2}=2.5, C_{S3}=10.0, C_{S4,5}=0.5, C_{S6}=1.0, C_{S7}=1.0, C_{S8}=3.0, C_{S9}=2.0, C_{S10}=2.0, C_{S11}=0.2, and C_{S12}=0.7.

As an example, the β-LysAsn(145)His sequence of hemoglobin is not in an α-helix or in a loop between two βsheets, so S₁ through S₄=0, S_{S}=1. There is one hydrogen bond to the amide side chain nitrogen and one other to be broken to form the imide, but there are none to the amide carboxyl or the backbone nitrogen, so S₆=0, S₇ =1, S₈=0, and S₉=1. This Asn is near the carboxyl end of the chain and one residue from an α-helix on the amino side, so S₁₀=0, S₁₁=1, and S₁₂=5. The GlyLysAsnHisGly half life⁶ is 10.5 days. Therefore, C_{D}=(0.01)(10.5)e−^{(0.48)[(1)(1)+(2)(1)+(2)(10)+(0.2)(4)]}=(0.105)e^{(0.48)(5.8)}=(0.105)(16.184)=1.70.

C_{D} is multiplied by 100 to give the predicted Tris deamidation half-time in days for the amide.

Results for Asn are greater than 95% correct in predicting the fastest amide in a protein. It is also applicable to Gln.

It is also likely that isomerization of Asp and Glu can be modeled with the same procedure. Primary rate data on Asp and Glu isomerization or a correction factor to be applied to the Asn and Gln data is needed in order to do this.

### Conclusions:

Three different types of modifications that can be used in the engineering of deamidation and/or isomerization rates of amides and possibly acids have been invented. These are:
1. Modification of the residues or residue-like structures on either side of the amide—principally the one on the right (carboxyl side).
2. Modification of the amide—specifically Asn to Gln or Gln to Asn, but other types of modification can also be used, especially in the case of structures that are similar, but not a perfect match to those found in peptides, hormones, and proteins.
3. Modification of the three-dimensional environment around the amide. The necessary modifications can be determined from the three-dimensional deamidation prediction method. Each of the S parameters describes a quantitative addition to the reaction activation energy. Removal or addition of one or more of these elements will change the rate accordingly.

At least two types of deamidation are present. The ones on which this method is based, and which are most prevalent for amides with half-times less than a few hundred days, depending on conditions and providing especially catalytic ions are not present, are most strongly effected by the structure to the right of the amide (e.g. in the sequence GlyXxx(Amide/Acid)YyyGly the identity of Yyy is the most important factor). Also present is at least one more mechanism that is usually slower and has different sequence dependence. It is possible that this dependence as well as the left hand structure dependence (Xxx in the sequence GlyXxx(Amide/Acid)YyyGly) can also be modeled with a similar system, but this has not yet been demonstrated.

1. A method that is useful for quantitatively changing deamidation rates of Asn and Gln residues in peptides and proteins or in other molecules that contain these amides, including hormones and drugs and modifications of peptides, proteins, hormones and drugs.
(a) This can be done through substitution of the carboxyl-side residue to the amide with another residue, using the experimentally determined values in Tables 1 and 2.
(b) For example, suppose an investigator is confronted with the sequence -IleAsnAla- in a particular protein. From Table 1, he knows that the deamidation rate is 25.9 days under the conditions in the table. If a deamidation rate of, for example, 150 days is more desirable, an examination of the table shows that this can be done by changing the sequence to -IleAsnLeu-, which has a half-time of 154 days.
(c) Table 1 is to be used for Asn sequences and Table 2 for Gln sequences.

2. A method that is useful for quantitatively changing deamidation rates of Asn and Gln residues in peptides and proteins or in other molecules that contain these amides, including hormones and drugs and modifications of peptides, proteins, hormones and drugs.
(a) This can be done through substitution of the amino-side residue to the amide with another residue, using the experimentally determined values in Tables 1 and 2.
(b) For example, suppose an investigator is confronted with the sequence -IleAsnLeu- in a particular protein. From Table 1, he knows that the deamidation rate is 154 days under the conditions in the table. If a deamidation rate of, for example, 100 days is more desirable, an examination of the table shows that this can be done by changing the sequence to -GlyAsnLeu-, which Table 1 shows, has a half-time of 104 days.
(c) Table 1 is to be used for Asn sequences and Table 2 for Gln sequences.

3. A method that is useful for quantitatively changing deamidation rates of amides residues in peptides and proteins or in other molecules that contain these amides, including hormones and drugs and modifications of peptides, proteins, hormones and drugs through the swapping of Asn for Gln or of Gln for Asn.
(a) This is quantitatively done by reference to both Tables 1 and 2.
(b) For example, suppose an investigator is designing a drug for which a part of the structure contains the sequence -LeuAsnGly-. He can tell from Table 1 that the half-time is likely to be about 1 day (unless it is suppressed by three-dimensional structure) and wishes to slow deamidation down to improve the drugs shelf-life. Table 2 shows that this can be done simply by substituting the sequence -LeuGlnGly- which now has a half-time of 670 days.

4. A method that is useful for quantitatively changing deamidation rates of Asn and Gln residues peptides and proteins or in other molecules that contain these amides, including hormones and drugs and modifications of peptides, proteins, hormones and drugs, through modification of molecular structures nearby the amide in space, but not part of the residues immediately to the carboxyl side or amino side of the amide, as determined by a three-dimensional prediction procedure.
(a) This procedure identifies certain structural components that can have an effect on the deamidation rate as set out in paragraphs [0034] to [0057].
(b) For example, the beta-Lys-Asn145-His sequence of hemoglobin is not in an alpha-helix or in a loop between two beta sheets, so S1 through S4=0, S5=1. There is one hydrogen bond to the amide side chain nitrogen and one other to be broken to form the imide, but there are none to the amide carboxyl or the backbone nitrogen, so S6=0, S7=1, S8=0, and S9=1. This Asn is near the carboxyl end of the chain and one residue from an alpha-helix on the amino side, so S10=0, S11=1, and S12=5. From Table 1, the primary sequence half-time is 1-0.5 days. Therefore CD=(0.01)(10.5)e^{(0.48)[(1)(1)+(2)(1)+(2)(1-0)+(0.2)(4)]}=(0.105)e^{(0.48)(5.8)}=(0.105)(16.184)=1.70. The predicted half-time is (100)CD so the half-time is estimated to be 170 days.
If an investigator wishes to change this half-time to, for example, around 100 days, he can see from the parameters used to predict the rate that one possibility is to eliminate the amide side chain nitrogen hydrogen bond. Thus S7=0 and he can calculate CD=(0.01)(10.5)e^{(0.48)[(2)(1)+(2)(1-0)+(0.2)(4)]}=(0.105)e^{(0.48)(4.8)}=(0.105)(10.014)=1.05. The predicted half-time is now (100)CD so the estimated half-time is now 105 days.

5. A method that is useful for quantitative modification of the rate of isomerization, for Asn, and Gln residues using the technique outlined in paragraphs [0034] to [0057].
(a) For example, it is known that isomerization and deamidation occur through the same mechanisms. Thus the isomerization of Asn or Gln cannot take place without deamidation occurring. If it is found that ½ of deamidation for a particular peptide is accompanied by isomerization, then an investigator would know that the deamidation of, for example, the sequence -AlaAsnAla- in a drug with no three-dimensional interference, would have a primary half-time (using Table 1) of 22.5 days and that the isomerization half-time would be 45 days. If it is desirable to change this rate to about 75 days, he can do this by redesigning the drug to include a hydrogen bond in the S7 position and the isomerization rate would now be (45)e^{(0.48)(1)}=73 days.

6. A method that is useful for quantitative modification of the rate of isomerization, for Asp, and Glu residues using the technique outlined in paragraphs [0034] to [0057].
(a) For example, the rates of isomerization for Glu and Asp are about 100 fold slower than for deamidation. If one wishes to know the rate of isomerization of, for example, a peptide containing the sequence -GlyAspAla-, reference to Table 1 shows that the half-time for deamidation of this sequence is 21.1 days. If there are no three-dimensional constraints the method reduces to this half-time multiplied by 100 for isomerization. Thus the isomerization half-time can be estimated as (21.1)(100)=2100 days. In order to slow this reaction down, an investigator could add a hydrogen bond in the C₅₉ category. This would slow it down by a factor of e^{(0.48)(2)}=2.6, so the new half-time would be (2.6)(2100)=5460 days.

7. A method that is useful for quantitative modification of the rate of chain cleavage, for Asn, Gln, Asp, and Glu residues using the technique outlined in paragraphs [0034] to [0057].
(a) For example, if a particular sequence in a protein, for example, -LeuAsnPro- is found to undergo cleavage with a half-time of 200 days, and an investigator wishes to speed this reaction up to around 100 days, he may be able to do so by removing a hydrogen bond. If C₅₉ is still 2.0 and one hydrogen bond exists in this position, removing it would speed the reaction up by a factor of e^{(0.48)(2)}=2.6 and the new half-time is 77 days.
